(19) United States
(12) Patent Application Publication
(10) Pub. No.: US 2010/0223685 A1
(43) Pub. Date: Sep. 2, 2010

(54) IL-21 RECEPTOR KNOCKOUT ANIMAL AND METHODS OF USE THEREOF

(76) Inventors: Marion KASAIAN, Charlestown, MA (US); Matthew WHITTERS, Hudson, MA (US); Andrea WURSTER, Arlington, MA (US); Mary COLLINS, Natick, MA (US); Deborah YOUNG, Melrose, MA (US); Michael GRUSBY, Newton, MA (US)

Correspondence Address: FITZPATRICK CELLA HARPER & SCINTO, 1290 Avenue of the Americas, NEW YORK, NY, 10104-3800, US

(21) Appl. No.: 12/683,117

(22) Filed: Jan. 6, 2010

Related U.S. Application Data
(62) Division of application No. 10/418,450, filed on Apr. 17, 2003, now abandoned.
(60) Provisional application No. 60/373,746, filed on Apr. 17, 2002.

(51) Int. Cl. A01K 67/00; C12Q 1/68; C12N 5/10; C12N 15/63

(52) U.S. Cl. 800/14; 435/6; 435/354; 800/18; 800/22; 800/25

(57) ABSTRACT

A transgenic non-human mammal with a disruption in its IL-21 receptor gene is provided, along with methods of using the transgenic non-human mammal.

### RELATED APPLICATIONS

This application is a divisional of U.S. application Ser. No. 10/418,450, filed Apr. 17, 2003, which claims priority to U.S. Provisional Application No. 60/373,746, filed Apr. 17, 2002. The contents of both applications are incorporated herein by reference in their entirety.

### FIELD OF THE INVENTION

The invention relates generally to transgenic animals and more particularly to transgenic animals with disruptions in the Interleukin-21 Receptor (IL-21R) gene.

### BACKGROUND OF THE INVENTION

The vertebrate immune system can be functionally divided into cell compartments providing both adaptive immunity and innate immunity.

Innate immunity is characterized by a lack of specific recognition of particular foreign agents. This type of immunity provides initial protection against foreign pathogens, such as viruses, bacteria and protozoa. Cells of the innate immune system do not specifically recognize foreign pathogens but are nevertheless adept at distinguishing normal, healthy host cells from abnormal—infected, damaged or transformed host cells—and selectively killing these abnormal cells. One type of cell involved in the innate immunity is the natural killer (NK) cell. The ability of NK cells to efficiently distinguish healthy host “self” cells from infected or otherwise “nonself” cells and to effectively kill the latter accounts for the significant role these lymphoid cells play in tissue graft and transplant rejection.

Adaptive immunity is directed against specific molecules and is enhanced by re-exposure. Adaptive immunity is mediated by lymphocytes that synthesize cell-surface receptors or secrete proteins that bind specifically to foreign molecules. This response also recognizes and kills invading organisms such as bacteria, viruses, and fungi, but effects a cascade of molecular and cellular events that ultimately results in the humoral and cell-mediated immune response. This pathway of the immune defense generally commences with the trapping of the antigen by antigen presenting cells (APCS), such as dendritic cells and macrophages. These cells are capable of internalizing, partially digesting, and displaying the “processed” antigen on their cell surfaces. The adaptive immune response of the vertebrate system relies, in part, on cells of the lymphoid line. These cells include B cells, which give rise to soluble antibodies, and T cells, including T helper, T suppressor, and cytotoxic T cells.

Agents involved in modulating the transition between innate and adaptive immunity are incompletely understood. These agents include, e.g., cytokines.

### SUMMARY OF THE INVENTION

The invention is based in part on the discovery of a transgenic mouse lacking a functional receptor for the cytokine IL-21. The transgenic mouse is useful, inter alia, for evaluating the role of IL-21 in modulating immune responses, including the transition between innate and adaptive immunity.

Accordingly, in one aspect the invention provides a transgenic non-human mammal whose genome includes a disruption of an IL-21 receptor (IL-21R) gene such that the mammal lacks or has reduced levels of functional IL-21 receptor polypeptide.

In some embodiments, thymocytes from the transgenic mammal do not proliferate when contacted with IL-21.

In some embodiments, the mammal is a rodent (e.g., a mouse or a rat).

In some embodiments, the IL-21R gene encodes a IL-21R polypeptide includes the amino acid sequence of SEQ ID NO:2.

In one embodiment, one allele of the IL-21R gene in the mammal is disrupted. In other embodiments, two alleles of the IL-21R gene of the mammal are disrupted.

In some embodiments, the disruption of the IL-21R gene is located on a homologue of human chromosome 16p12.

In some embodiments, the disruption of the IL-21 R gene includes a substitution of an exon of the IL-21R gene with an exogenous nucleic acid sequence.

Also provided by the invention is a cultured cell isolated from a transgenic mammal that has a disruption of an IL-21R gene. The genome of the cell includes a disruption of a IL-21R gene.

In another aspect, the invention provides an isolated mammalian cell whose genome includes a disruption of an IL-21 receptor (IL-21R) gene such that the cell lacks or has reduced levels of functional IL-21 receptor polypeptide.

In some embodiments, the cell is an embryonic stem cell. In some embodiments, the embryonic stem cell is a murine embryonic stem cell, e.g., a murine stem cell is derived from a mouse strain of C57BL/6 origin. An example of such a stem cell is a J12 embryonic stem cell.

In a further aspect, the invention provides a method of producing a non-human mammal with a disruption in a IL-21 receptor (IL-21R) gene. The method includes introducing a transgenic non-human embryonic stem cell whose genome includes a disruption of an IL-21 receptor (IL-21R) gene such into a blastocyst, thereby forming a chimeric blastocyst and introducing the chimeric blastocyst into the uterus of a pseudopregnant mammal. At least one transgenic progeny is recovered from the pseudopregnant mammal, wherein the genome of the progeny includes a disruption of the IL-21 R gene such that the progeny lacks or has reduced levels of functional IL-21 R polypeptide.

In some embodiments, the transgenic non-human embryonic stem cell is prepared by introducing a targeting vector which disrupts the IL-21R gene into a mammalian embryonic stem cell, thereby producing a transgenic embryonic stem cell with the disrupted IL-21R gene, and selecting the transgenic embryonic stem cell whose genome includes the disrupted IL-21R gene.

In some embodiments, the method further includes breeding the transgenic mammal with a second mammal to generate F1 progeny having a heterozygous disruption of the IL-21R gene, thereby expanding the population of mammals having a heterozygous disruption of the IL-21R gene and crossbreeding the F1 progeny to produce a transgenic mammal that contains a homozygous disruption of the IL-21R gene.

In some embodiments, the transgenic mammal is a rodent, such as a mouse, rat, hamster or guinea pig.

Also provided by the invention is method for identifying the role of IL-21 in a biological process. The method includes providing a transgenic cell whose genome includes a disruption of an IL-21 receptor (IL-21R) gene such that the cell lacks or has reduced levels of functional IL-21 receptor polypeptide and measuring one or more properties associated with the biological process. The properties are compared to a reference cell whose genome does not have a disruption in an IL-21R gene. A difference in the one or more properties indicates IL-21 affects the biological process.

In a further aspect, the invention provides a method for identifying the role of IL-21 in a biological process. The method includes providing a transgenic non-human mammal whose genome includes a disruption of an IL-21 receptor (IL-21R) gene such that the cell lacks or has reduced levels of functional IL-21 receptor polypeptide and measuring one or more properties associated with the biological process. The properties are compared to a reference mammal whose genome does not have a disruption in an IL-21R gene. A difference in the properties indicates IL-21 affects the biological process.

Also within the invention is a method for determining whether a test agent selectively modulates IL-21 receptor (IL-21R) activity. The method includes administering a test agent to a first non-human mammal and a second non-human mammal, wherein the first non-human mammal includes functional wild-type IL-21R polypeptide and wherein the genome of the second non-human transgenic mammal includes a disruption of its endogenous IL-21R genes such that the mammal lacks functional IL-21R polypeptide and comparing a biological response elicited the agent in the first mammal and the second mammal. An alteration in the response indicates the test agent selectively modulates the IL-21 receptor.

In another aspect, the invention provides a method for promoting the transition from innate to adaptive immunity in a subject. The method include administering to the subject an agent that increases IL-21 levels or activity in the subject, thereby promoting the transition from innate to adaptive immunity in the subject. The agent can be, e.g., an IL-21 protein, a nucleic acid encoding an IL-21 protein, or an agonistic antibody to an IL-21 Receptor. In some embodiments, the IL-21 protein or nucleic acid sequence has the amino acid sequence or nucleic acid sequence of a human IL-21 protein or polynucleotide.

In some embodiments, the method further includes administering to the subject an agent that inhibits expression of an IL-15 gene or activity of an IL-15 polypeptide.

Also within the invention is a method for promoting antigen-specific T cell activation in a subject. The method includes contacting an NK cell population from the subject with an agent that increases IL-21 levels or activity in the subject in an amount sufficient to induce adaptive immunity in the subject. The method include administering to the subject an agent that increases IL-21 levels or activity in the subject, thereby promoting the transition from innate to adaptive immunity in the subject.

In various embodiments, the cells are provided in vitro, in vivo, or ex vivo. Cells provided in vitro or ex vivo can optionally be administered to the subject after they have been contacted with the agent that increases IL-21 levels or activity.

The agent can be, e.g., an IL-21 protein or a nucleic acid encoding an IL-21 protein. In some embodiments, the IL-21 protein or nucleic acid sequence has the amino acid sequence or nucleic acid sequence of a human IL-21 protein or polynucleotide.

In some embodiments, the method further includes administering to the subject an IL-15 an agent that inhibits expression of an IL-15 gene or activity of an IL-15 polypeptide.

In another aspect, the invention provides a method for inhibiting the transition from innate to adaptive immunity in a subject by administering to the subject an agent that decreases IL-21 levels or activity in the subject. The agent can be, e.g., a polypeptide that includes the extracellular region of an IL-21R polypeptide fused to a second polypeptide, such as one comprising an Fc region of an IgG1 polypeptide. In other embodiments, the agent is an IL-21 antibody or IL-21 receptor antibody. In some embodiments, the method further includes administering to the subject an agent that increases IL-15 levels or activity.

Also within the invention is method for inhibiting antigen-specific T cell activation in a subject. The method includes contacting an NK cell population from the subject with an agent that decreases IL-21 levels or activity in the subject in an amount sufficient to inhibit antigen-specific T cell activation in the subject. The agent can be, e.g., a polypeptide that includes the extracellular region of an IL-21R polypeptide fused to a second polypeptide, such as one comprising an Fc region of an IgG1 polypeptide. In other embodiments, the agent is an IL-21 antibody or IL-21 receptor antibody. In some embodiments, the method further includes administering to the subject an agent that increases IL-15 levels or activity.

Also provided by the invention is a method of inhibiting expansion of an NK cell population. The method includes contacting an NK cell population in need thereof with IL-21 in an amount sufficient to inhibit expansion of the NK cell population.

In some embodiments, the NK cell population is a resting NK cell population.

In some embodiments, addition of IL-21 does not inhibit activation of the resting NK cell population.

In some embodiments, the method further includes administering to the subject an agent that decreases IL-15 expression or activity.

In a further aspect, the invention provides a method of enhancing a T cell response to an alloantigen. The method includes providing a cell population of T cells and antigen presenting cells and culturing the cell population in the presence of IL-21, thereby enhancing the response of the T cells to the alloantigen.

In some embodiments, the method further includes administering to the subject an agent that decreases IL-15 expression or activity.

In some embodiments, the cell population is provided in vitro.

In some embodiments, the cell population is provided in vivo.

In a still further aspect, the invention provides a method of inhibiting a T cell response to an alloantigen. The method includes providing a cell population of T cells and antigen presenting cells and culturing the cell population in the presence of an agent that decreases IL-21 expression or activity, thereby inhibiting the response of the T cells to the alloantigens.

In some embodiments, the cell population is provided in vitro.

In some embodiments, the cell population is provided in vivo.

In some embodiments, the method further includes administering to the subject an agent that increases IL-15 levels or activities.

In another aspect, the invention provides a method of inhibiting an immune response, the method includes administering to a subject in need thereof an agent that inhibits IL-15 expression or activity and an agent that decreases expression or activity of IL-21 in the subject, thereby inhibiting an immune response in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIGS. 1A-1C depict studies examining functional inactivation of the IL-21R gene. (A) The structure of the wild-type allele, recombinant gene, and knock-out construct are shown at top, middle, and bottom, respectively. The knock-out construct, consisting of a neomycin resistance cassette flanked by appropriate linkers for homologous recombination, was targeted to replace the IL-21R exon 1 sequence. (B) Thymocytes isolated from wild-type (4611 and 4613) or IL-21R−/− (4615 and 4616) mice; or (C) lymph node cells pooled from wild-type or IL-21R−/− mice were incubated 3 days with media (no cytokine), IL-21 (30 U/ml), or COS mock control on anti-CD3− coated plates, and 3H-thymidine incorporation assayed over the final 5 hours.

FIGS. 2A-2C depict studies showing that IL-21R−/− mice have normal NK cell number and display full NK cell activation in vivo and in vitro. (A) Flow cytometric analysis of spleen lymphocytes from wild-type or IL-21R−/− mice. NK cells, identified as NK1.1+/CD3−, are boxed. (B) Wild-type or IL-21R−/− mice were injected i.p. with poly I:C or PBS control, and spleens harvested 1.5 days later. (C) Spleen cells isolated from wild-type or IL-21R−/− mice were treated in vitro with IL-15 (50 ng/ml) for 7 days, then used as effectors in a 5 hour 51-Cr release assay against YAC-1 targets. Cells were pooled from 2-3 mice per group.

FIGS. 3A-3C depict studies showing that IL-21 prevents IL-15-induced expansion of resting NK cells. Spleen cells from wild-type or IL-21R−/− mice were cultured for 7 days with IL-15 (10 ng/ml)+COS mock control or IL-15+IL-21 (12.5 U/ml). (A) NK and T cell subsets were identified as (NK1.1+/CD3−) or (NK1.1−/CD3+), respectively, by flow cytometry. Results shown are averages of 5-11 experiments, each done with lymphocytes pooled from 2-3 spleens (wild-type Day 0 and treated, and IL-21R−/− Day 0), or with a single pool of 2-3 spleens (IL-21R−/− treated). (B) Typical flow cytometric analysis of cells from wild-type mice on day 7 of culture with IL-15+COS mock control or IL-15+IL-21. NK cells (NK1.1+/CD3−) are boxed. (C) Spleen cells were cultured with IL-15 (10 ng/ml) in the presence of 6.25 U/ml IL-21 (Δ) or an equivalent volume of COS mock control (). At the time points indicated, the percentage of NK cells (NK1.1+/CD3−) was determined by flow cytometry. (D) Spleen cells were cultured with the indicated concentration of IL-15 in the presence of 6.25 U/ml IL-21 (Δ) or an equivalent volume of COS mock control (). On day 7, the percentage of NK cells (NK1.1+/CD3−) was determined by flow cytometry.

FIGS. 4A-4C depict studies showing that IL-21 boosts NK cytotoxicity in spleen cells activated with poly I:C in vivo or IL-15 in vitro, but does not induce activation of resting NK cells. (A, D) Resting Cells: Spleen cells isolated from wild-type (A) or IL-21R−/− (D) mice were treated for 2-3 days with IL-15 (10 ng/ml)+COS mock control ( ), COS mock control only ( ), IL-21 (12.5 U/ml) only (Δ), or (A) IL-15+IL-21 (▴). (B, E) Poly I:C-Activated Cells: Spleen cells isolated from wild-type (B) or IL-21R−/− (E) mice 1.5 days post-i.p. administration of poly I:C were cultured 2 days with the indicated treatment. (C, F) IL-15-activated Cells: Spleen cells isolated from untreated wild-type (C) or IL-21R−/− (F) mice were cultured 7 days with IL-15 (10 ng/ml), then washed and restimulated for 2 days with the treatments as described above. Data are shown as mean+/−s.d. of replicate wells in a 5 hour 51Cr-release assay against YAC-1 targets. Effector: target ratios were corrected for the percentage of NK1.1+/CD3− cells in each culture, identified by flow cytometry.

FIG. 5 depicts studies showing that IL-21 boosts IFNγ production by IL-15-activated spleen cells, but blocks their growth. Spleen cells isolated from wild-type (A) or IL-21R−/− (B) mice were cultured 7 days with IL-15 (10 ng/ml), then restimulated for 2 days with IL-15 (10 ng/ml)+COS mock control ( ), COS mock control only ( ), IL-15 (10 ng/ml)+IL-21 (12.5 U/ml) (▴), or IL-21 (12.5 U/ml) only (Δ). For determination of IFNγ production, cells were washed free of cytokine and challenged for 24 hours with the indicated concentration of murine IL-12. Data are shown as mean+/−s.d. of IFNγ levels in replicate culture wells. (C) Cell concentrations from cultures activated 7 days with IL-15 and restimulated 2 or 5 days with the indicated treatment, at the doses indicated above. The NK cell concentration on day 7 of culture with IL-15 (prior to restimulation) was 4.1×10⁶/ml. (D) Apoptosis in spleen cell cultures expanded for 5 days with IL-15 (10 ng/ml) and restimulated for 1 or 2 days with agents as described above. At each time point, cells were stained for surface expression of NK1.1 and CD3, and intracellular TUNEL. Data are shown for NK cells gated as NK1.1+/CD3−.

FIGS. 6A and 6B depict studies showing that IL-21 prevents IL-15-induced expansion of CD44^{hu} CD8+ T cells. Spleen cells isolated from IL-21R−/− or wild-type mice (BALB/c×C57BL/6 background) were cultured 7 days in IL-15 (50 ng/ml)+COS mock supernatant or IL-15+IL-21 (25 U/ml). (A) Cell surface marker expression was analyzed on day 0 and on day 7 of culture. CD44, CD119, and CD132 were analyzed on gated CD8+CD3+ cells. CD122 and CD25 were analyzed on total CD3+ cells. Appropriate gates were established using three-color flow cytometry. (B) Spleen cells grown 7 days with IL-15+COS mock supernatant or IL-1530 IL-21 were washed free of cytokine, then re-plated with the indicated concentration of IL-2 or IL-15. 3H-thymidine incorporation was assayed over 24 hours.

FIGS. 7A-7C depict studies showing that IL-21 enhances T cell proliferation and activation in response to alloantigen. (A) T cell enriched populations from lymph nodes of wild-type and IL-21R−/− mice (BALB/c×C57BL/6 background) were cultured 3-4 days with irradiated allogeneic spleen cells, in the presence of no cytokine, IL-21 (10 U/ml), or COS Mock control. 3H-thymidine incorporation was assayed over the final 12 hours of culture. (B) Cells isolated from cultures of primary allogeneic stimulation were assayed for cytotoxicity against allogeneic target cells in a 4 hour 51-Cr release assay. Data are corrected for % CD8+ T cells under each priming condition. (C) IFNγ production was assayed from T cells “primed” as indicated, following a 40 hour secondary stimulation with fresh allogeneic spleen cells and no added cytokine

### DETAILED DESCRIPTION OF THE INVENTION

Provided by the invention is a transgenic non-human mammal that includes disruptions in the IL-21R gene. The term mammal includes an individual animal in all stages of development, including embryonic and fetal stages. A “transgenic mammal” is an animal containing one or more cells bearing genetic information received, directly or indirectly, by deliberate genetic manipulation at a subcellular level, such as by microinjection or infection with recombinant virus. This introduced DNA molecule can be integrated within a chromosome, or it can be extra-chromosomally replicating DNA. Unless otherwise noted or understood from the context of the description of an animal, the term “transgenic mammal” as used herein refers to a transgenic mammal in which the genetic information was introduced into a germ line cell, thereby conferring the ability to transfer the information to offspring. If offspring possess some or all of the genetic information, then they, too, are transgenic mammals. The genetic information is typically provided in the form of a transgene carried by the transgenic mammal.

IL-21R nucleic acids can be used to generate transgenic animals or site specific gene modifications in cell lines. Transgenic animals may be made through homologous recombination, where the normal IL-21R locus is altered. Alternatively, a nucleic acid construct is randomly integrated into the genome, Vectors for stable integration include plasmids, retroviruses and other animal viruses, YACS, and the like.

The modified cells or animals are useful in the study of IL-21 and/or IL-21R function and regulation. For example, the role of IL-21 in the transition between innate and adaptive immunity can be assessed as described in the Examples below. The role of IL-21 in a biological (including a diseased process) can be monitored in a transgenic animal in which the IL-21R gene has been disrupted. Generation of IL-21R deficient transgenic non-human animals, including mice, also aids in defining the in vivo function(s) of IL-21R. Such IL-21R null animals can be used as a strain for the insertion of human IL-21R genes, and provides an animal model useful in the design and assessment of various approaches to modulating IL-21R activity and expression. Such modified transgenic non-human animals can also be used as a source of cells for cell culture. These cells can be used for corresponding in vitro studies of IL-21R expression, activity and the modulation thereof. These cells can also be used to monitor the expression of other proteins, particularly those involved in modulating an immune response, in cells in which the IL-21R gene is disrupted.

Animals with disrupted IL-21 receptor genes, especially mice, provide a convenient model system for the study of disease, immune disorders, and of diseases associated with immune disorders. Suitable disorders include, e.g., various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV, hepatitis viruses, herpes viruses) as well as bacterial or fungal infections (including mycobacteria, Leishmania spp., malaria spp. and various fungal infections such as candidiasis), and autoimmune disorders. Autoimmune disorders include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Additional conditions include allergic reactions and conditions, such as asthma (particularly allergic asthma) or other respiratory problems.

Animals suitable for transgenic experiments can be obtained from standard commercial sources. These include animals such as mice and rats for testing of genetic manipulation procedures, as well as larger animals such as pigs, cows, sheep, goats, and other animals that have been genetically engineered using techniques known to those skilled in the art. These techniques are briefly summarized below based principally on manipulation of mice and rats.

DNA constructs for homologous recombination will comprise at least a portion of the IL-21R gene with the desired genetic modification, and will include regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line can be used, or ES cells may be obtained freshly from a host, e.g. mouse, rat, guinea pig, etc. Such cells are grown on an appropriate fibroblast-feeder layer or grown in the presence of leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected.

The methods for evaluating the targeted recombination events as well as the resulting knockout mice are readily available and known in the art. Such methods include, but are not limited to DNA (Southern) hybridization to detect the targeted allele, polymerase chain reaction (PCR), polyacrylamide gel electrophoresis (PAGE) and Western blots to detect DNA, RNA and protein.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc.

The transgenic animal with disruptions in the IL-21R genes can be introduced into other genetic backgrounds to study various diseased states. Animal models include murine experimental autoimmune encephalitis, systemic lupus erythematosis in MRL/lpr/lpr mice or NZB hybrid mice, murine autoimmune collagen arthritis (including DBA1 mice), diabetes mellitus in NOD mice and BB rats, and murine experimental myasthenia gravis (see Paul ed., Fundamental Immunology, Raven Press, New York, 1989, pp. 840-856). Progeny from such crosses with desired genotypes can then be repeatedly crossed as desired to produce a desired strain.

As used herein, a “targeted gene” or “Knockout” (KO) is a DNA sequence introduced into the germline of a non-human animal by way of human intervention, including but not limited to, the methods described herein. The targeted genes of the invention include nucleic acid sequences which are designed to specifically alter cognate endogenous alleles.

The invention provides also methods and compositions for modulating immune responses, or various aspects of an immune response, by modulating the level or activity of the cytokine IL-21 in a subject.

Mice lacking functional IL-21R (IL-21R−/−) have been used to address the influence of a lack of IL-21 signaling on innate and adaptive immunity. IL-21R−/− mice had normal lymphocyte compartments and no NK cell deficiency, an unexpected finding given the previously described role of IL-21 in human NK cell maturation (Parrish-Novak et al., Nature 408: 57-63, 2000). Cells from these mice did not display any response to IL-21 detectable in these assays, including effects on T cell proliferation, NK cell activation and expansion, and cytokine receptor expression The findings in mouse reveal that innate NK cell responses, and the cytokine-driven TCR-independent outgrowth of CD44^{hi} CD8+ T cells, were antagonized by IL-21, whereas antigen-driven T activation in an allogeneic MIR was stimulated. As a product of activated T lymphocytes that acts to limit ongoing NK cell expansion while promoting antigen-specific T cell-mediated immunity, IL-21 may be a key element in the transition between innate and adaptive immune responses.

Innate immune mechanisms shape the adaptive cellular responses that follow. In turn, adaptive immunity likely feeds back to limit ongoing innate responses, but the mechanisms by which this occurs are poorly understood. During acute pathogen infections, the NK cell response begins within hours, as IFNα/β, IL-12, IL-15, and IL-18 generated by infected cells stimulate NK cytotoxicity, cytokine production, and expansion (Biron et al., Annu. Rev. Immunol. 17: 189-220, 1999). Along with enhanced effector function, the maturation of NK cells is ultimately accompanied by their terminal differentiation. An emerging view (Loza et al., Nature Immunology 2: 917-924, 2001) supports a sequential process of NK cell development in the human system resulting in generation of committed IFNγ-producing effector NK cells, whose subsequent terminal differentiation coincides with abatement of the innate response. Although cytokine regulation of initial NK cell recruitment and activation has been intensively studied (Biron et al., Annu. Rev. Immunol. 17: 189-220, 1999), the signals responsible for resolution of this response remain to be defined. The concordance of decreased NK cell responses with the emergence of antigen-specific T cells makes it likely that T cell-derived factors influence the final steps of NK cell maturation.

IL-21 has been found to inhibit the IL-15-dependent expansion of both resting NK cells and those that had undergone prior stimulation. On previously activated NK cells, IL-21 induces apoptosis that was accompanied by a burst of cytotoxicity and IFNγ production. IL-21 also blocked the IL-15-driven, TCR-independent expansion of CD44^{hi} CD8+ cells. In contrast, IL-21 enhances proliferation, cytotoxic activation, and IFNγ production by antigen-specific T cells. None of these effects are seen in IL-21R−/− mice, confirming the requirement for this receptor chain in mediating cellular responses to IL-21.

IL-21R−/− mice have normal numbers of mature peripheral NK cells, capable of full activation. This result is surprising in view of findings by Parrish-Novak et al. (Nature 408: 57-63, 2000) that IL-21 potentiates IL-15- and Flt3L-induced NK cell expansion from progenitors in human bone marrow. Indeed, a role in NK cell development is difficult to reconcile with activated, mature T cells being the only known source of IL-21 (Parrish-Novak et al., Nature 408: 57-63, 2000), as the maturation of NK cells in T cell-deficient athymic (Nassiry et al., Nat. Immun. Cell Growth Regul. 6: 250-259, 1987), RAG−/− (Shinkai et al., Cell 68:855-867, 1992), and SCID (Dorshkind et al., J. Immunol. 134: 3798-3801, 1985) mice argues against any critical requirement for a T cell-derived factor in NK cell development. Previous studies have shown that mice rendered deficient in IL-15 (Kennedy et al., J. Exp. Med. 191: 771-780, 2000) and its receptor components (Di Santo et al., Proc. Natl. Acad. Sci. USA 92: 377-81, 1995; Suzuki et al., J. Exp. Med. 185: 499-505, 1997; Lodolce et al., Immunity 9: 669-76, 1998), or Flt3L (McKenna et al., Blood 95: 3489-3497, 2000) have profoundly reduced NK cell numbers, underscoring the critical role of these agents in NK cell development. Other cytokines, IL-2 and ckit ligand (SCF), play an auxiliary role. Both can synergize with Flt3L to drive NK cell development from bone marrow progenitors in vitro (Muench et al., Exp. Hematology. 28:961-973, 2000; Mrozek et al., Blood 87: 2632-2640, 1996), or when administered in vivo (Fehniger et al., Blood 90: 3647-3653, 1997), but mice lacking IL-2 (Kundig et al., Science 262: 1059-61, 1993) or ckit (W/Wv mice) (Seaman et al., Exp. Hematol. 9, 691-696: 1981; Colucci et al., Blood 95: 984-91, 2000) have NK cells, albeit at reduced number and activity. Vosshenrich and Di Santo (2001) have speculated that because IL-21R utilizes γc (Asao et al., J. Immunol. 167, 1-5, 2001), and because mice lacking γc have even an more profound reduction in NK cell numbers (Di Santo et al., Proc. Natl. Acad. Sci. USA 92: 377-81, 1995) than those lacking IL-15Rα (Lodolce et al., Immunity 9: 669-76, 1998), IL-21 could be a key factor in promoting NK cell development in vivo. Although a role in human NK cell development cannot be excluded, the finding of normal NK cell numbers and full cytolytic potential in IL-21R−/− mice indicates that IL-21, acting through this receptor chain, is neither essential nor regulatory for NK cell maturation in mice.

Nevertheless, IL-21 is able to influence NK cell viability and function, in a manner that discriminated between resting and activated cells. Although RNAse protection analysis confirms expression of IL-21R chain transcripts in both resting and activated NK cells, IL-21 enhances effector function only when used to restimulate NK cells following their initial activation in vivo with the poly I:C, or in vitro with IL-15. In contrast, IL-21 inhibits the IL-15-mediated expansion of NK cells under all conditions tested. In this regard, IL-21 is distinct from the related cytokines, IL-2 and IL-15, both of which are able to induce proliferation and cytolytic activation of resting NK cells (Carson et al., J. Clin. Invest. 99: 937-943, 1997 1997; London et al., J. Immunol. 137:3845-3854, 1986). Cells from IL-21R−/− mice were fully able to undergo initial activation in response to poly I:C in vivo or IL-15 in vitro, but showed no enhancement of function upon restimulation with IL-21. The ability of IL-21 to enhance effector function only of previously activated NK cells may reflect differential expression of alternative IL-21 receptor chains, signaling molecules, or receptor-induced transcription factors upon initial NK cell activation. Although the basis for the differential IL-21 responsiveness of resting vs. activated NK cells remains to be determined, the potential of IL-21 to discriminate between them may be important in vivo. A recent report by Yokoyama and colleagues showed that murine NK cells responding early in the course of virus infection are activated nonspecifically, whereas those that persist late into infection require more specific activation signals (Dokun et al., Nature Immunology 2: 951-956, 2001). The selectivity of IL-21 for NK cells that have undergone an initial response could be one mechanism by which those cells that persist late into infection continue to receive activation signals, while the ability of IL-21 to block expansion of resting NK cells could be a mechanism to prevent further recruitment of resting NK cells to the response.

For both resting and activated NK cells, IL-21 alone does not sustain viability. IL-21 also blocks survival but not cytotoxicity induced by IL-15. The observation that growth inhibition is absent in IL-21R−/− mice argues that IL-21R is required to mediate this effect. Because IL-21 blocked NK cell growth in response to IL-2 as well as IL-15 (data not shown), and all three cytokines utilize the γc receptor chain (Asao et al., J. Immunol. 167, 1-5, 2001), one possible explanation is that IL-21 blocked growth effects by competing for a limited pool of γc receptor chains. This type of inhibition can be overcome by addition of higher amounts of IL-15. In the studies described in the Examples below, however, IL-21 has been found to block NK cell outgrowth at all doses of IL-15 to which the NK cells responded, inconsistent with a model of simple competition between IL-21 and IL-15 for γc chain interactions. An additional possibility is that the pro-apoptotic effects of IL-21 prevail over the growth-promoting effects of IL-15, even though the pathways leading to apoptotic vs. growth signals are separate, as has been outlined for IL-2 effects on T cells (Van Parijs et al., 1999).

Following an initial activation event in vivo or in vitro, subsequent challenge with IL-21 greatly enhances both NK cell cytolytic activity per cell and IFNγ production. This functional activation is necessarily transient, because even as IL-21 promoted NK cell effector responses, it antagonized viability through induction of apoptosis. Given the rapid, potent, and relatively nonspecific nature of their responses, there is a clear biological imperative to have control over expansion of NK cells. This normally occurs during the course of an immune response when abatement of NK cell activation coincides with the emergence of antigen-specific T cells (Biron et al., Ann. Rev. Immunol. 17: 189-220, 1999). While inhibitory receptors can prevent inappropriate activation, few agents have been described that reduce NK cell proliferation once it has been initiated. The T cell-dependent release of TGFβ is such one mechanism (Su et al., J. Immunol. 151:4874-4890, 1993), but clearly others exist (Pierson et al., Blood 87: 180-189, 1996). Recently, Loza et al., Nature Immunology 2: 917-924, 2001) have demonstrated that human NK cells undergo step-wise maturation from IL13-producing (NK2) to IFNγ-producing (NK1) effectors, whose activation is followed by terminal differentiation and apoptosis. Generation of IFNγ-producing NK cells was stimulated by the monocyte-derived factor, IL-12, and slowed by the T cell-derived agent, IL4 (Loza et al., Nature Immunology 2: 917-924, 2001), suggesting the possibility that emergence of activated T cells feeds back to limit recruitment of NK cells into the immune response. By blocking responsiveness of NK cells to the growth-promoting effects of IL-15, while acting as a potent IFNγ-inducer, IL-21 may be a key regulator of NK cell functional status by promoting the terminal steps of NK cell maturation.

Antagonism of IL-15 function is also apparent in IL-21 effects on CD8+ T cells expressing a memory-associated phenotype, CD44^{hi}. As described by Sprent and colleagues, a small percentage of cells which can respond to IL-15 in the absence of TCR signaling arises following contact with antigen in the mouse, and persists in vivo in the absence of ongoing antigenic stimulation (Zhang et al., 1998). Treatment with IL-15 in vivo or in vitro selectively induces proliferation of CD44^{hi}CD8+ T cells in an antigen non-specific manner (Sprent et al., Current Opin. Immunol. 13: 248-254, 2001). Although cells with this phenotype initially arise primarily following encounter with antigen (Sprent et al., Current Opin. Immunol. 13: 248-254, 2001), their subsequent TCR-independent, cytokine-mediated re-activation displays functional characteristics allied with the innate immune response. They undergo bystander proliferation in vivo in response to type I interferon or poly I:C (Tough et al., Science 272: 1947-1950, 1996), expand within the first 2 days of virus infection (Turner et al., J. Immunol. 167: 2753-2758, 2001), proliferate in response to LPS administration (Tough et al., J. Exp. Med. 185: 2089-2094: 1997), and produce IFNγ in vivo within hours of bacterial infection (Lertmemongkolcahi et al., J. Immunol. 166: 1097-1105, 2001). Addition of IL-21 has been found to inhibit the IL-15-mediated in vitro expansion of CD44^{hi} CD8+ T cells from wild-type but not IL-21R−/− spleens. It has yet to be determined whether this decreased expansion is due to prevention of IL-15-induced proliferation, or is a pro-apoptotic effect of IL-21 similar to that of IL-2, which may override the growth signals of IL-15 to induce apoptosis in T cells (Li et al., Nature Med. 7:114-118, 2001), including CD44^{hi} CD8+ T cells (Ku et al., Science 288: 675-78, 2000). Decreased IL-15 responsiveness of CD44^{hi} CD8+ T cells mediated by the T cell activation product, IL-21 (Parrish-Novak et al., Nature 408: 57-63, 2000), may be one mechanism whereby cytokine-driven “bystander” T cell proliferation is reduced once specific immunity emerges. A recent report that CD44^{hi} CD8+ T cells undergo apoptosis-mediated attrition upon induction of TCR-mediated anti-viral immunity in LCMV infection (McNally et al., J. Virol. 75: 5965-5976, 2001) is consistent with this hypothesis, and suggests a role for IL-21.

Whereas IL-21 fails to support expansion of either NK cells or cytokine-activated, TCR-independent CD44^{hi} CD8+ T cells, it delivers a potent TCR-dependent accessory signal for T cell responses to alloantigen. Both the allospecific proliferation of freshly isolated T cells and secondary effector responses, including cytotoxicity and IFNγ production, are stimulated by IL-21. Potentiation of TCR-mediated responses is also apparent in the enhanced proliferation of both thymic and peripheral T cells in response to sub-optimal concentrations of anti-CD3 with IL-21. To underscore the essential role of IL-21 in these costimulatory responses, cells from IL-21R−/− mice were used in an allogeneic MLR, and did not display IL-21-mediated potentiation of allogeneic T cell responses. While this might indicate that IL-21R−/− cells are somehow impaired in antigen specificity or in interaction with APCs, the finding that these cells have full lytic capacity against allogeneic targets confirms their antigen recognition potential. These observations implicate IL-21 as a potent inducer of CD8+ effector mechanisms in response to allogeneic stimulation, and suggest that in the presence of IL-21, the expansion and effector mechanisms of antigen-specific T cells would be greatly enhanced, even as NK and antigen non-specific T cell responses were diminished.

In summary, these studies show that IL-21R is not required for the development of NK cells in the mouse, but is necessary to mediate all NK cell and T cell responses to IL-21 that were examined. IL-21 reduced survival of both resting and activated NK cells, even while promoting the effector function of those NK cells that had undergone initial activation in vivo or in vitro. IL-21 also inhibited the proliferation of TCR-independent CD44^{hi} CD8+ T cells in response to IL-15. In contrast, it provided a potent accessory signal for anti-CD3− or antigen-dependent T cell proliferation and effector function. During the course of an immune response, the development and mobilization of antigen-specific T cells coincides with diminished innate responses. This transition, which involves a decrease in NK cell numbers and concomitant T cell expansion (Biron et al., Ann. Rev. Immunol. 17: 189-220, 1999), may be initiated by the presence of activated, mature T cells. As a product of activated T cells that functions to augment antigen-specific T cell responses while antagonizing NK cell survival, IL-21 may be a key facilitator of this transition.

Promotion of the transition from innate to adaptive immunity is indicated in situations in which a heightened antigen-specific immune response is desired. In contrast, inhibition of the transition from innate to adaptive immunity is desired in situations in which a heightened innate immune response is desired and/or suppression of an antigen-specific immune response is desired.

Agents for Promoting the Transition from Innate to Adaptive Immunity

An agent that increases IL-21 levels or activity in the subject is administered when promotion of the transition from innate to adaptive immunity is desired in a subject. Thus, the agent can be, e.g., an IL-21 polypeptide itself, a fragment of an IL-21 polypeptide, an IL-21 R-binding fragment of an IL-21 polypeptide, a nucleic acid encoding an IL-21 polypeptide, or a nucleic acid encoding an IL-21R-binding fragment of an IL-21 polypeptide. Amino acid sequences of IL-21 polypeptides, as well as nucleic acids encoding these sequences are publicly known. Human and murine IL-21 polypeptide and nucleotide sequences are disclosed in Parrish-Novick et al., Nature 408:57-63, 2000. The nucleotide sequence and amino acid sequence of a human IL-21 polypeptide sequence is also available at Genbank Acc. No. X_{—}011082.

Agents for Inhibiting the Transition from Innate to Adaptive Immunity

To promote innate immunity and/or inhibit the transition between innate and adaptive immunity, an inhibitor of IL-21 expression or activity is administered to a subject. For inhibiting the expression of IL-21 the inhibitor can be, an antisense IL-21 RNA molecule, or an interfering RNA derived from an IL-21 RNA. Suitable agents for inhibiting activity of an IL-21 polypeptide include, e.g., an antibody to an IL-21 or IL-21R polypeptide, Another type of inhibitor is a polypeptide that includes an IL-21 binding portion of an IL-21 receptor polypeptide. Such inhibitors can be constructed using IL-21R polypeptide and nucleic acid sequence information that is known in the art, coupled with standard methods for constructing antibodies and inhibitor polypeptides that include extracellular portions of cytokine receptors.

Human and murine IL-21R polypeptide sequences, and the nucleic acids encoding these polypeptides are disclosed in, e.g., U.S. Pat. No. 6,057,128, Ozaki et al., Proc. Nat. Acad. Sci. USA 97:11439-44, 2000, Parrish-Novak et al., Nature 408:57-63, 2000. A cDNA encoding a human IL-21R was also deposited with the American Type Culture Collection on Mar. 10, 1998, as accession number ATCC 98687.

A murine IL-21R nucleotide sequence and the polypeptide encoded by the nucleic acid sequence are provided below:

```
(SEQ ID NO: 1)
      1 cagctgtctg cccacttctc ctgtggtgtg cctcacggtc acttgcttgt ctgaccgcaa
    61 gtctgcccat ccctggggca gccaactggc ctcagcccgt gccccaggcg tgccctgtct
  121 ctgtctggct gccccagccc tactgtcttc ctctgtgtag gctctgccca gatgcccggc
  181 tggtcctcag cctcaggact atctcagcag tgactcccct gattctggac ttgcacctga
  241 ctgaactcct gcccacctca aaccttcacc tcccaccacc accactccga gtcccgctgt
  301 gactcccacg cccaggagac cacccaagtg ccccagccta aagaatggct ttctgagaaa
  361 gaccctgaag gagtaggtct gggacacagc atgccccggg gcccagtggc tgccttactc
  421 ctgctgattc tccatggagc ttggagctgc ctggacctca cttgctacac tgactacctc
  481 tggaccatca cctgtgtcct ggagacacgg agccccaacc ccagcatact cagtctcacc
  541 tggcaagatg aatatgagga acttcaggac caagagacct tctgcagcct acacaggtct
  601 ggccacaaca ccacacatat atggtacacg tgccatatgc gcttgtctca attcctgtcc
  661 gatgaagttt tcattgtcaa tgtgacggac cagtctggca acaactccca agagtgtggc
  721 agctttgtcc tggctgagag catcaaacca gctcccccct tgaacgtgac tgtggccttc
  781 tcaggacgct atgatatctc ctgggactca gcttatgacg aaccctccaa ctacgtgctg
  841 aggggcaagc tacaatatga gctgcagtat cggaacctca gagaccccta tgctgtgagg
  901 ccggtgacca agctgatctc agtggactca agaaacgtct ctcttctccc tgaagagttc
  961 cacaaagatt ctagctacca gctgcaggtg cgggcagcgc ctcagccagg cacttcattc
1021 agggggacct ggagtgagtg gagtgacccc gtcatctttc agacccaggc tggggagccc
1081 gaggcaggct gggaccctca catgctgctg ctcctggctg tcttgatcat tgtcctggtt
1141 ttcatgggtc tgaagatcca cctgccttgg aggctatgga aaaagatatg ggcaccagtg
1201 cccacccctg agagtttctt ccagcccctg tacagggagc acagcgggaa cttcaagaaa
1261 tgggttaata cccctttcac ggcctccagc atagagttgg tgccacagag ttccacaaca
1321 acatcagcct tacatctgtc attgtatcca gccaaggaga agaagttccc ggggctgccg
1381 ggtctggaag agcaactgga gtgtgatgga atgtctgagc ctggtcactg gtgcataatc
1441 cccttggcag ctggccaagc ggtctcagcc tacagtgagg agagagaccg gccatatggt
1501 ctggtgtcca ttgacacagt gactgtggga gatgcagagg gcctgtgtgt ctggccctgt
1561 agctgtgagg atgatggcta tccagccatg aacctggatg ctggccgaga gtctggccct
1621 aattcagagg atctgctctt ggtcacagac cctgcttttc tgtcttgcgg ctgtgtctca
1681 ggtagtggtc tcaggcttgg aggctcccca ggcagcctac tggacaggtt gaggctgtca
1741 tttgcaaagg aaggggactg gacagcagac ccaacctgga gaactgggtc cccaggaggg
1801 ggctctgaga gtgaagcagg ttccccccct ggtctggaca tggacacatt tgacagtggc
1861 tttgcaggtt cagactgtgg cagccccgtg gagactgatg aaggaccccc tcgaagctat
1921 ctccgccagt gggtggtcag gacccctcca cctgtggaca gtggagccca gagcagctag
1981 catataataa ccagctatag tgagaagagg cctctgagcc tggcatttac agtgtgaaca
2041 tgtaggggtg tgtgtgtgtg tgtgtgtgtg tgtgtgtgtg tgtgtgtgtg tgtgtgtgtg
2101 tgtgtgtctt gggttgtgtg ttagcacatc catgttggga tttggtctgt tgctatgtat
2161 tgtaatgcta aattctctac ccaaagttct aggcctacga gtgaattctc atgtttacaa
2221 acttgctgtg taaaccttgt tccttaattt aataccattg gttaaataaa attggctgca
2281 accaattact ggagggatta gaggtagggg gcttttgagt tacctgtttg gagatggaga
2341 aggagagagg agagaccaag aggagaagga ggaaggagag gagaggagag gagaggagag
2401 gagaggagag gagaggagag gagaggagag gagaggctgc cgtgagggga gagggaccat
2461 gagcctgtgg ccaggagaaa cagcaagtat ctggggtaca ctggtgagga ggtggccagg
2521 ccagcagtta gaagagtaga ttaggggtga cctccagtat ttgtcaaagc caattaaaat
2581 aacaaaaaaa aaaaaaaa
(SEQ ID NO: 2)
MPRGPVAALLLLILHGAWSCLDLTCYTDYLWTITCVLETRSPNPSILSLTWQDEYEELQDQETFCSLHRS
GHNTTHIWYTCHMRLSQFLSDEVFIVNVTDQSGNNSQECGSFVLAESIKPAPPLNVTVAFSGRYDISWDS
AYDEPSNYVLRGKLQYELQYRNLRDPYAVRPVTKLISVDSRNVSLLPEEFHKDSSYQLQVRAAPQPGTSF
RGTWSEWSDPVIFQTQAGEPEAGWDPHMLLLLAVLIIVLVFMGLKIHLPWRLWKKIWAPVPTPESFFQPL
YREHSGNFKKWVNTPFTASSIELVPQSSTTTSALHLSLYPAKEKKFPGLPGLEEQLECDGMSEPGHWCII
PLAAGQAVSAYSEERDRPYGLVSIDTVTVGDAEGLCVWPCSCEDDGYPAMNLDAGRESGPNSEDLLLVTD
PAFLSCGCVSGSGLRLGGSPGSLLDRLRLSFAKEGDWTADPTWRTGSPGGGSESEAGSPPGLDMDTFDSG
FAGSDCGSPVETDEGPPRSYLRQWVVRTPPPVDSGAQSS
```

Techniques for generating anti-IL-21 and IL-21R sequences are known in the art. Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference).

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies).

If desired, a humanized antibody or human antibody to IL-21 or to an IL-21R is used to inhibit the transition from innate to adaptive immunity. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that primarily the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad. Sci. USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Another type of agent is a protein (preferably a fusion protein) that includes a portion of an IL-21 polypeptide or IL-21R polypeptide that, when introduced into a host, results in diminished IL-21 activity. On type of fusion protein includes an extracellular portion of an IL-21R polypeptide. For example, the agent can be a fusion protein that includes the extracellular, IL-21 binding region of the IL-21R linked to a second polypeptide. A preferred polypeptide is an Fc portion of a human IgG1 polypeptide. In one embodiment, the Fc component contains the CH₂ domain, the CH₃ domain and hinge region, but not the CH₁ domain of IgG1. IL-21R fusion proteins are described in Carter et al., US Patent Application 20030049798.

### Assessing Innate and Adaptive Immune Responses

Innate immunity and adaptive immunity can be measured using methods known in the art. In one embodiment, innate immunity is characterized by NK cell activity. Thus, an innate immune response can be assessed by measuring interferon gamma (IFN-γ) production and effector function of NK cells using methods known in the art, some of which are discussed below and illustrated in the examples below.

Adaptive immunity can be assessed using methods known in the art. In one embodiment, adaptive immunity is measured using an allogenic mixed lymphocyte reactions assaying lytic activity and IFN γ as illustrated in the examples below. Mixed lymphocyte reaction (MLR) assays additionally include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Assays for T-cell or thymocyte proliferation additionally include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E.e.a. Coligan eds. Vol 1 pp. 3.12.1-3.12.14, John Wiley and Sons, Toronto. 1994; and Measurement of mouse and human Interferon.gamma., Schreiber, R. D. In Current Protocols in Immunology. J. E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S, and Lipsky, P. E. In Current Protocols in Immunology. J. E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J. E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11-Bennett, F., Giannotti, J., Clark, S. C. and Turner, K. J. In Current Protocols in Immunology. J. E.e.a. Coligan eds. Vol 1 pp. 6.15.1 John Wiley and Sons, Toronto. 1991; Measurement of mouse and human Interleukin 9-Ciarletta, A., Giannotti, J., Clark, S. C. and Turner, K. J. In Current Protocols in Immunology. J. E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. U.S.A. 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E.e.a. Coligan eds. Vol 1 pp. 3.8.1-3.8.16, John Wiley and Sons, Toronto. 1994.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. U.S.A. 88:7548-7551, 1991.

### Pharmaceutical Compositions

The agents discussed above can be provided any form suitable for administration to a subject. The subject can be, e.g., a human, a non-human primate (including a chimpanzee or gorilla), a cow, pig, horse, goat, sheep, cat, or rodent (such as a rat or mouse).

A pharmaceutical composition containing an agent may be in the form of a liposome in which isolated IL-21R protein is combined, in addition to other pharmaceutically acceptable carriers, with amphipathic agents such as lipids which exist in aggregated form as micelles, insoluble monolayers, liquid crystals, or lamellar layers which in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions.

Administration of the agent can be carried out in a variety of conventional ways, such as oral ingestion, inhalation, or cutaneous, subcutaneous, or intravenous injection. Intravenous administration to the patient is preferred.

When a therapeutically effective amount of the agent is administered orally, it is conveniently delivered in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% IL-21R protein, and preferably from about 25 to 90% the agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the agent, and preferably from about 1 to 50% the agent.

When a therapeutically effective amount of the agent is administered by intravenous, cutaneous or subcutaneous injection, the agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to the agent an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additive known to those of skill in the art.

The amount of the agent in the pharmaceutical composition will depend upon the nature and severity of the condition being treated, and on the nature of prior treatments which the patient has undergone. Ultimately, the attending physician will decide the amount of the agent with which to treat each individual patient. Initially, the attending physician will administer low doses of the agent and observe the patient's response. Larger doses of the agent may be administered until the optimal therapeutic effect is obtained for the patient, and at that point the dosage is not generally increased further. It is contemplated that the various pharmaceutical compositions used to practice the method of the present invention should contain about 0.1 μg to about 100 mg of the agent per kg body weight.

The duration of intravenous therapy using the pharmaceutical composition will vary depending on the severity of the disease being treated and the condition and potential idiosyncratic response of each individual patient. It is contemplated that the duration of each application of the agent will be in the range of 12 to 24 hours of continuous intravenous administration. Ultimately the attending physician will decide on the appropriate duration of intravenous therapy using the pharmaceutical composition of the present invention. The polynucleotide and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

The invention will be further illustrated in the following non-limiting examples.

### Example 1

### Experimental Procedures for Examples 2-7

Materials and methods used in the examples provided in Examples 2-7 are provided below.

### Targeting the IL-21R Gene by Homologous Recombination and Generation of IL-21R−/− Mice

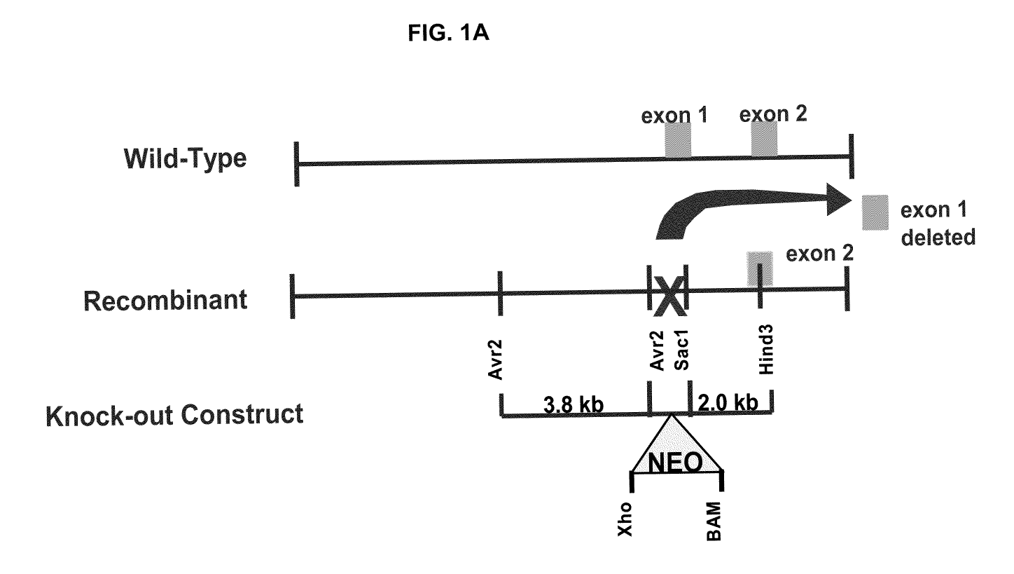

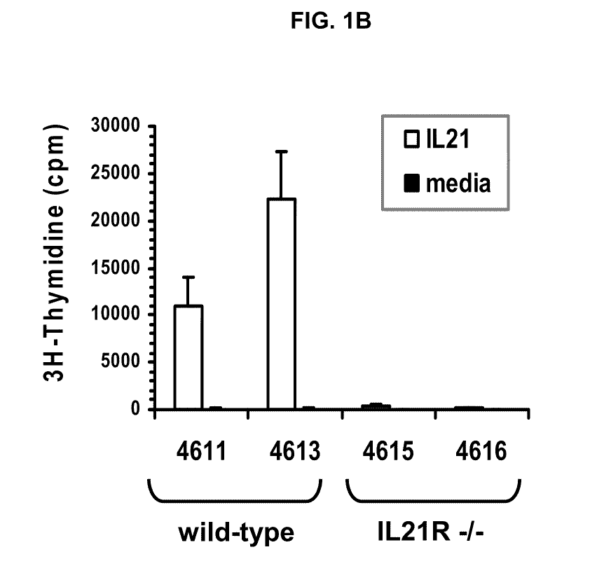

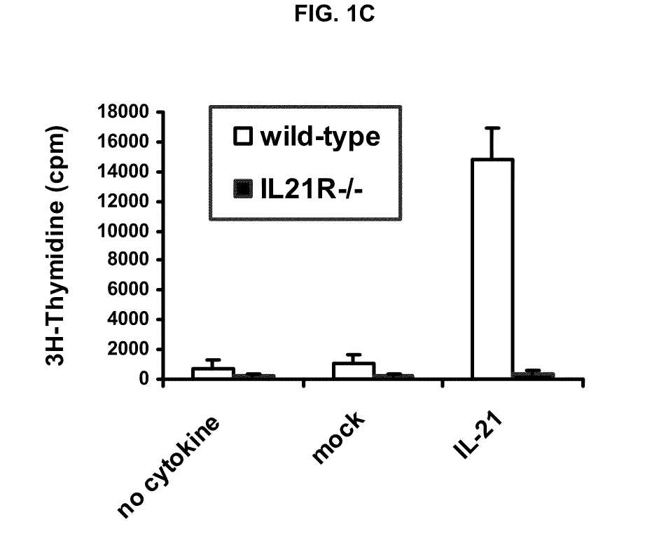

A 400 by Sph1/EcoRV cDNA fragment of IL-21R corresponding to 5′ coding regions (2-135aa) was used as a probe to screen a Stratagene (La Jolla, Calif.) C57BL/6 mouse liver genomic library for the IL-21R gene. Four clones were isolated from screening 1×10⁶ colony forming units. One clone of approximately 13 kb was partially sequenced and found to contain 2 exons corresponding to the signal sequence region of murine IL-21R. The first leader exon (MPRGPVAALLLLILHG) (SEQ ID NO:3) was targeted for deletion by replacing it with a neomycin resistance cassette (FIG. 1A). A 3.8 kb Avr2 fragment 5′ to the first leader exon and a 2.0 kb Sac1/Hind3 intronic fragment 3′ to the first leader exon were ligated, 5′ and 3′, respectively, via linkers (Avr2/Xho1 and BamH1/Sac1) to the 1.1 kb Xho/Bam neomycin cassette and subcloned in to pTK(SK).

IL-21R−/− mice were generated by targeting the IL-21R gene in the J12 embryonic stem cell line (C57BL/6 origin), injecting clones into blastocysts and transferred to pseudopregnant BALB/c females. Resulting male chimeras were bred to BALB/c females and offspring were analyzed by PCR and Southern blotting for germline transmission of the mutant alleles. Mice heterozygous for the IL-21R mutation (IL-21R+/−) were intercrossed to yield homozygous offspring (IL-21R−/−) on the BALB/c×C57BL/6 background, and subsequently bred onto the C57BL/6 background. The lack of IL-21R expression in IL-21R−/− mice was confirmed by PCR analysis using total RNA isolated from tail bleeds and analysis of amplified products by Southern blot. Mice on the BALB/c×C57BL/6 background were used for initial characterization, and those on the C57BL/6 background were used for functional studies, unless otherwise noted. Data are presented for mice aged between 8 and 12 weeks.

### Murine IL-21

The 441 by coding region of mIL-21 cDNA, encoding a protein of 146 amino acids (Parrish-Novak et al., 2000) was inserted into the COS-1 expression vector, pEDΔc. COS transfectants were grown in DMEM containing 10% FBS in 10% CO₂. Cells were placed in serum-free DMEM for 30 hours post transfection and IL-21 containing supernatant was collected 24 and 48 hours later. The supernatant was concentrated 50× by Amicon filtration. PMSF and EDTA were added to prevent proteolysis. One unit of activity was defined as the concentration of supernatant required to induce 50% maximal proliferation of Ba/F3 cells transfected with IL-21R. When tested in the absence of exogenous IL-3, proliferation of these transfectants is IL-21-dependent. Mock transfected COS supernatant, prepared and concentrated in parallel with IL-21, was used as a control.

### NK Cell Activation In Vitro

Spleen cell suspensions were depleted of RBC with ammonium chloride, and plated in RPMI containing 10% FBS, 50 U/ml penicillin, 50 μg/ml streptomycin, 2 mM L-glutamine, and 50 μg/ml β-mercaptoethanol with 10-50 ng/ml recombinant human IL-15 (R&D Systems, Minneapolis, Minn.), 12.5 U/ml recombinant mouse IL-21 or an equal volume of COS mock control supernatant. This dose of IL-21 had been shown to have maximal activity for the inhibition of NK cell outgrowth (data not shown). Cells were cultured at 5% CO₂ for 7 days, with a second dose of IL-15 and IL-21 or COS mock control added on day 4. On day 7, non-adherent and adherent cells were assayed for cytotoxicity, IFNγ production, or cell surface phenotype.

### NK Cell Activation In Vivo

Mice were injected i.p. with 0.15 ml PBS containing 1 mg/ml polyinosinic-polycytidylic acid (poly I:C; Sigma), or PBS control. Spleens were harvested 1.5 days later, and cells used in a 51Cr-release assay against YAC-1 targets.

### NK Cytotoxicity

YAC-1 target cells (American Type Culture Collection, Manassas, Va.) were incubated for 1 hour with sodium 51-chromate (20 μCi/1×10⁵ cells; New England Nuclear, Boston, Mass.), washed and plated with effector cells at the indicated effector: target cell (E:T) ratio. After 5 hour incubation at 37° C., supernatants were harvested, and radioactivity determined in a gamma counter. Maximum release was determined by lysing YAC-1 target cells with Triton X-100. Spontaneous release was determined as 51Cr released into the supernatant of YAC-1 targets incubated in the absence of effectors. Percent specific lysis was calculated according to the formula: (test−spontaneous release)/(total−spontaneous release)×100.

### IFNγ Production

Spleen cells treated with IL-15 or IL-21 as described above were washed and re-plated at 5×10⁵/ml for 24 hours with the indicated concentration of recombinant murine IL-12 (Genetics Institute, Cambridge, Mass.). IFNγ levels were assayed by Quantikine mouse IFNγ ELISA kit (R&D Systems; detection limit=10 pg/ml).

### Thymocyte Proliferation

Single cell thymocyte suspensions were cultured in DME containing 10% FBS, 50 U/ml penicillin, 50 μg/ml streptomycin, 2 mM L-glutamine, and 50 μg/ml β-mercaptoethanol, in the presence or absence of 25 U/ml IL-21. Cells were plated at 2×10⁵/well in 96-well flat-bottom plates coated with 1 μg/ml anti-CD3 (mAb 2C11; Pharmingen). On day 3, cultures were pulsed with 0.5 μCi/well 3H-thymidine (Amersham Biosciences, Piscataway, N.J.), and harvested 5 hours later onto glass fiber filter mats. ³H-thymidine incorporation was determined by liquid scintillation counting.

### Mixed Lymphocyte Reaction

T cells were purified from lymph nodes of wild-type or IL-21R−/− mice using negative selection columns (R&D Systems). For primary proliferation assays, T cells (2×10⁶/ml) were cultured in 96-well plates coated with 1 μg/ml anti-CD3 (mAb 2C11, Genetics Institute) or with erythrocyte-depleted, irradiated B10.Br splenocytes (3×10⁷/ml) and the indicated cytokine for 3 days, then pulsed for 12 hours with 1 μCi 3H-thymidine. For effector function assays, purified LN T cells (5×10⁵/ml) were primed with erythrocyte-depleted, irradiated B10.Br splenocytes (2×10⁶/ml) in the presence of the indicated cytokines: rhIL-2 (20 U/ml; R&D Systems), rmIL-21 (10 U/ml), or an equivalent volume of COS mock control supernatant. After 6-7 days, cells were harvested, washed and used in a 4 hour CTL assay with B10.Br or syngeneic spleen blasts as target cells. The splenic blasts prepared by 48 hour treatment of erythrocyte-depleted splenocytes with 10 μg/ml LPS and DXS (Sigma). Percent specific lysis was calculated as for NK cytotoxicity. For IFNγ production, T cells “primed” with alloantigen and the indicated cytokines were washed, counted, and restimulated (2.5×10⁵/ml) with irradiated B10.Br splenocytes (1×10⁶/ml) for 40 hours. Supernatants were assayed for IFNγ levels by ELISA (R&D Systems).

### Flow Cytometry and Quantitation of Lymphocyte Subsets

Cells were resuspended in PBS containing 1% BSA, and incubated 15 min., 4° C. with Fc block (PharMingen), followed by biotinylated antibody to various cell surface markers or appropriate isotype control (PharMingen). Cells were washed in the same buffer, then incubated 15 min., 4° C. with the appropriate FITC-labelled or PE-labelled antibody to cell surface markers or isotype control (PharMingen), and streptavidin-Red 670 (Gibco Life Technologies). Fluorescein TUNEL staining was performed using the In Situ Cell Death Detection Kit (Roche Diagnostics, Mannheim, Germany). Analysis was performed on a FACScan with CellQuest software (Becton-Dickinson, San Jose, Calif.). In all cases, viable cells were gated based on forward and side scatter. For quantitation of various cell subsets, the percentage of cells in that subset was determined by flow cytometry, and multiplied by the total number of lymphocytes per culture.

### Example 2

### Generation and Characterization of IL-21R−/− Mice

Mice were made genetically deficient in IL-21R (IL-21R−/−) as described above and outlined in FIG. 1A. IL-21R−/− mice were viable and fertile, and were bred on both BALB/c×C57BL/6 and C57BL/6 backgrounds. Adult IL-21R−/− mice had normal numbers of peripheral blood erythrocytes, monocytes, granulocytes, and lymphocytes. Phenotypic analysis of T cell, B cell, and monocyte populations in spleen, lymph node, and thymus showed no significant differences between IL-21R−/− and wild-type. In the serum, IL-21R−/− mice were found to have 3.3× lower levels of IgG1 (p<0.05), 2.2× lower IgG2b (p<0.05), and 2.8× higher levels of IgE (p<0.02) as compared to wild-type mice.

The absence of functional receptor was confirmed by lack of IL-21 responsiveness in cells isolated from IL-21R−/− mice. In accordance with the observations of Parrish-Novak et al. (2000), IL-21 enhanced the proliferation of thymocytes from wild-type, but not IL-21R−/− mice, in response to sub-optimal concentrations of anti-CD3 (FIG. 1B). In addition, IL-21 was found to enhance anti-CD3-responsiveness of lymph node T cells from wild-type but not IL-21R−/− mice (FIG. 1C). These observations support the functional inactivation of the IL-21R gene in IL-21R−/− mice.

### Example 3

### IL-21R−/− Mice have Normal NK Cell Numbers and Display Full NK Activation in Vivo and In Vitro

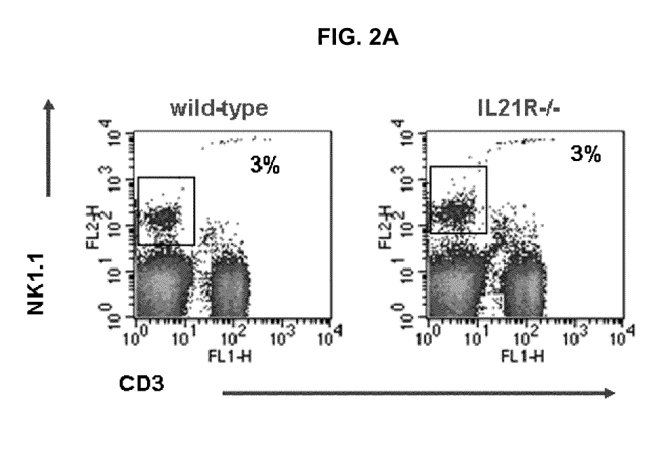

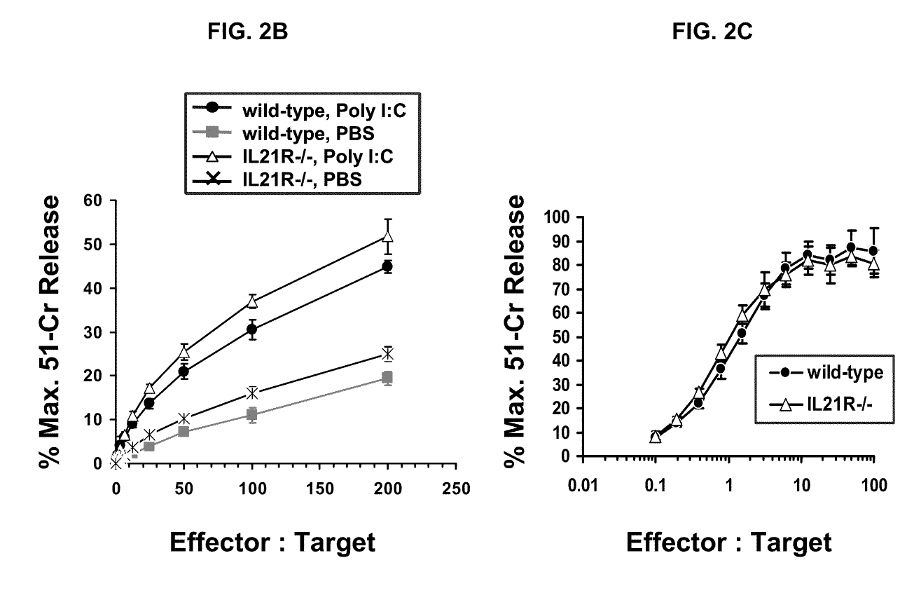

To determine if a lack of IL-21R affected the generation of mature NK cells, these cells were quantified in spleens of IL-21R−/− mice. Results with mice on BALB/c×C57BL/6 and C57BL/6 backgrounds were indistinguishable. Both the percentages (FIG. 2A) and the total numbers of NK cells (3.06+/−0.78×10⁶/spleen for wild-type and 3.77+/−0.91×10⁶/spleen for IL-21R−/− mice) were equivalent, indicating that IL-21R−/− mice had no intrinsic defect in the generation of phenotypically mature NK cells.

The ability of spleen NK cells IL-21R−/− mice to undergo activation in vivo and in vitro was examined. All functional studies were done with mice on the C57BL/6 background, unless otherwise noted. NK cells from IL-21R−/− mice were fully able to respond to poly I:C in vivo (FIG. 2B) or IL-15 in vitro (FIG. 2C) with induction of lytic activity that was indistinguishable from that found in NK cells from wild-type animals. This indicates that NK cells from IL-21R−/− mice are fully responsive to typical activating agents in vivo and in vitro.

### Example 4

### IL-21 Reduces IL-15-Mediated Expansion, but has No Effect on Activation of Resting NK Cells

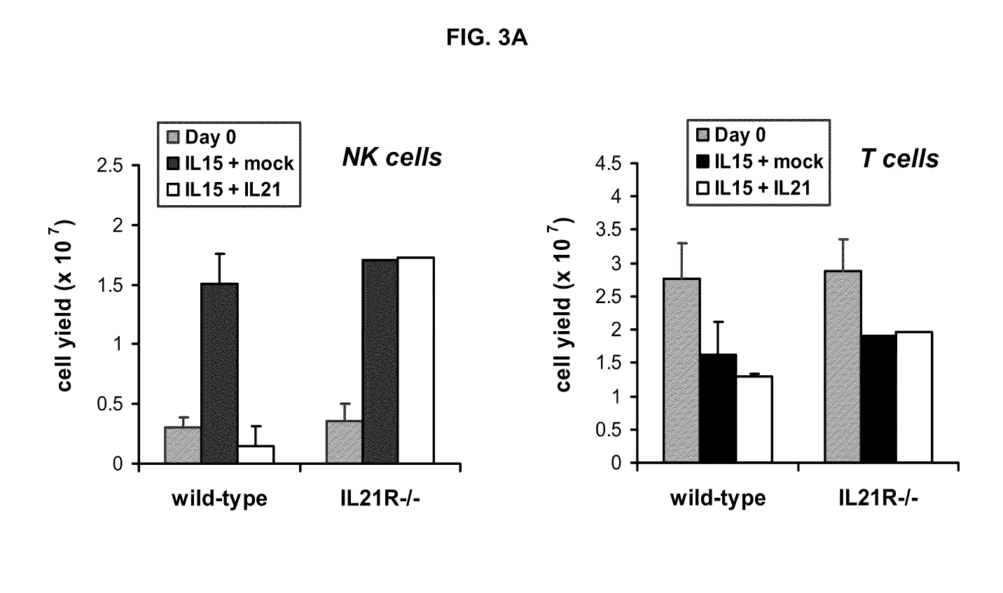

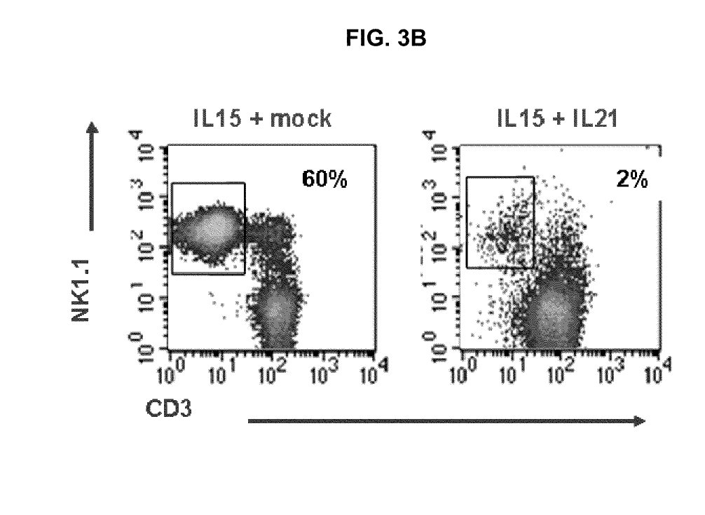

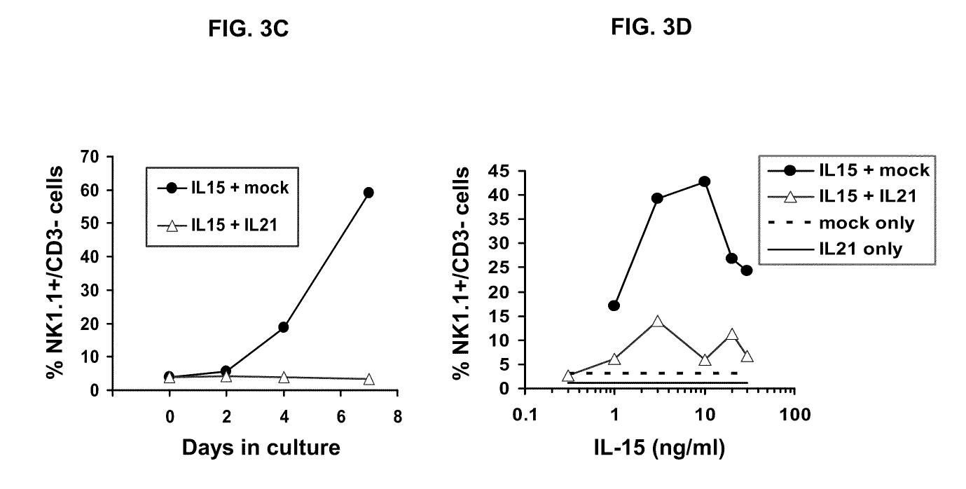

In addition to enhancing effector function, IL-15 enhances NK cell survival and proliferation (Carson et al., J. Clin. Invest. 99: 937-943, 1997), and these effects were comparable using splenic NK cells of wild-type and IL-21R−/− mice (FIG. 3A). IL-21 alone did not support expansion of NK cells in vitro. Therefore, to study the effects of IL-21 on NK cell outgrowth, IL-21 was used in conjunction with IL-15. For wild-type but not IL-21R−/− cells, addition of IL-21 inhibited IL-15-mediated NK cell expansion in a 7-day culture (FIG. 3A, 3B), but had no effect on total T cell numbers, which dropped ˜30% in these cytokine-driven, antigen-independent cultures (FIG. 3A). Similar findings were seen with IL-2-expanded cultures. Kinetic analysis revealed that IL-21 blocked IL-15-mediated NK cell proliferation throughout the culture period (FIG. 3C). Rather than shifting the effective dose of IL-15 required for NK cell expansion, IL-21 blocked NK cell outgrowth over the entire range of IL-15 concentrations to which the NK cells responded (FIG. 3D). Thus, IL-21 limits outgrowth of NK cells in response to IL-15.

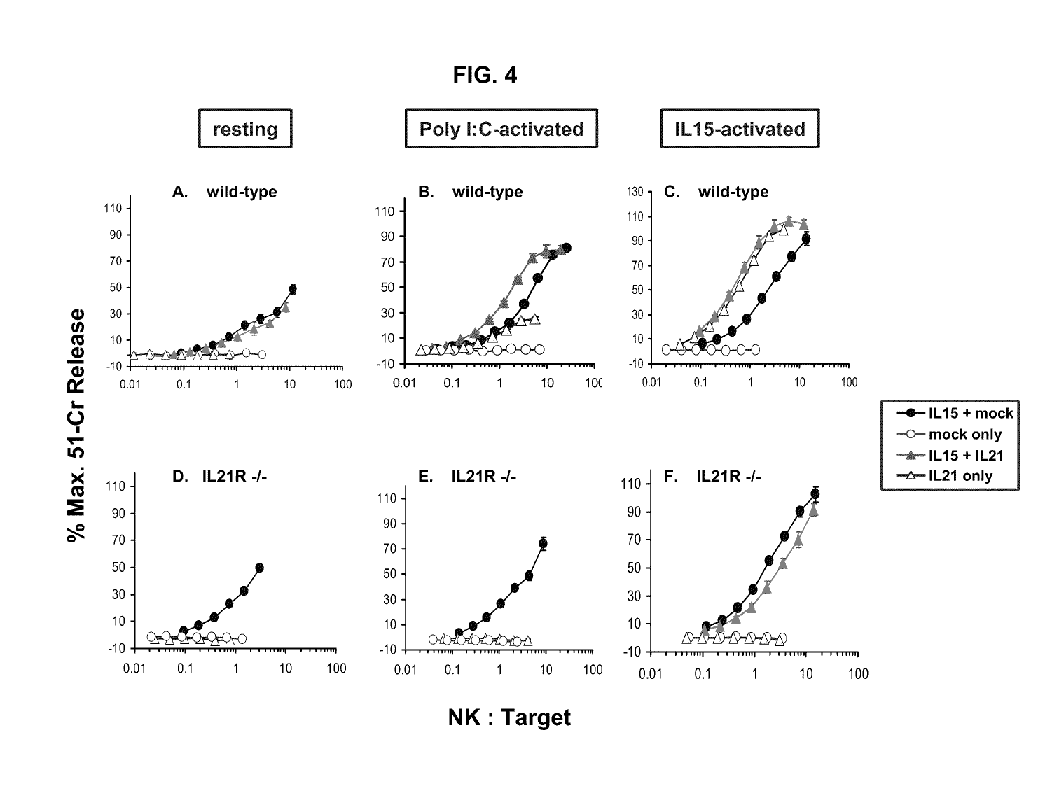

To further examine IL-21 effects on NK cell activation, freshly isolated murine splenocytes were cultured for 2-3 days in the presence of IL-21 and/or IL-15 and tested for cytotoxicity against NK-sensitive YAC-1 target cells. In response to IL-15, resting NK cells from both wild-type and IL-21R−/− mice became actively cytolytic (FIG. 2C and FIG. 4A, D). In contrast, IL-21 did not promote activation of resting NK cells (FIG. 4A,D) and had no effect on cytolytic potential per cell induced by IL-15 (FIG. 4A), although absolute NK cell numbers were greatly reduced in cultures containing IL-15+IL-21 (FIG. 3A). Taken together, these results indicate that IL-21 antagonizes IL-15-induced growth but not activation of resting NK cells.

### Example 5

### IL-21 Enhances Cytotoxicity of Previously Activated NK Cells, and Induces their Apoptosis

Parrish-Novak et al. (2000) found that IL-21 stimulates cytotoxicity of human NK cells enriched by positive selection from peripheral blood. The murine results presented above appeared contradictory, as no activation of murine splenic NK cells was seen in response to IL-21 (FIG. 4A). In an attempt to reconcile these observations, it was reasoned that human NK cells, continuously challenged with environmental agents, may exist in a heightened state of activation as compared to NK cells of a mouse residing in a specific pathogen-free facility. Therefore, IL-21 effects were examined on NK cells from mice that had been challenged in vivo with poly I:C to induce their activation.

Cells harvested from mice treated with poly I:C or PBS control were restimulated for 2-3 days in vitro with IL-15, IL-21, or COS mock control, then assayed for lytic activity. In contrast to its effects on NK cells from resting mice, IL-21 alone induced a high level of cytotoxic activity in NK cells from poly I:C-treated mice (FIG. 4B). In order to determine whether heightened IL-21 responsiveness would also follow NK cell activation in vitro, splenocytes were cultured for 7 days with IL-15, then restimulated for 2 days with IL-21, IL-15, or the combination. In this case, restimulation with either IL-21 or IL-15 alone greatly enhanced NK cytotolytic function (FIG. 4C). Results shown in FIGS. 4B and 4C, and other experiments suggest an additive effect of IL-15 and IL-21 on NK cell activation, with no indication of synergy. Cells from IL-21R−/− mice displayed full cytolytic activation with IL-15, but did not respond to IL-21 (FIGS. 4D-F). For these cells, IL-15+IL-21 produced no greater activation than IL-15 alone (FIG. 4F).

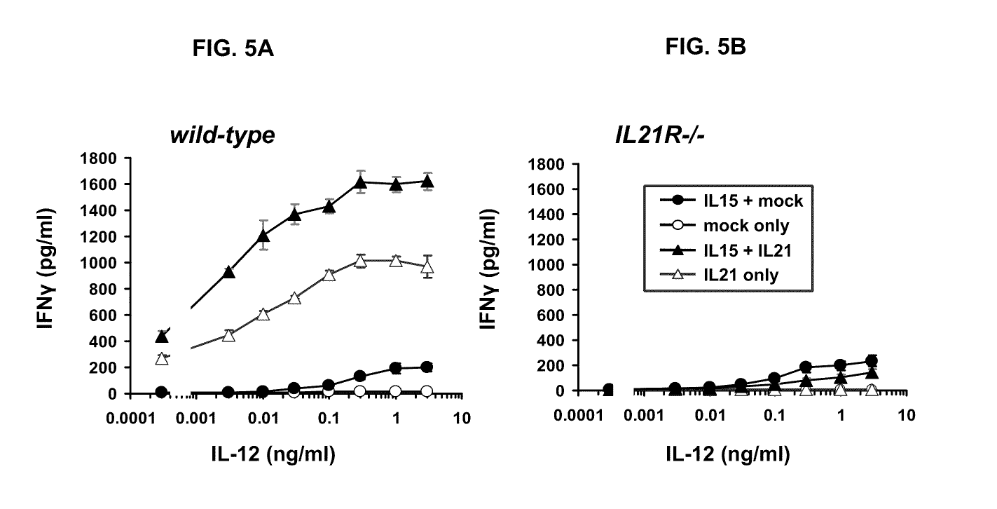

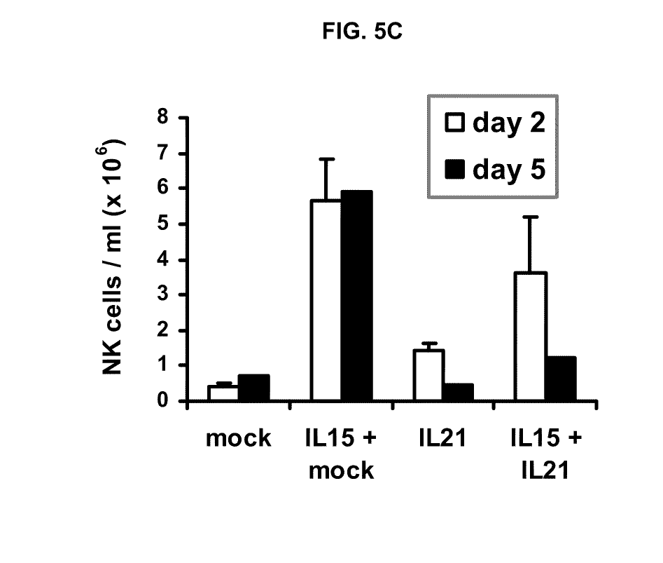

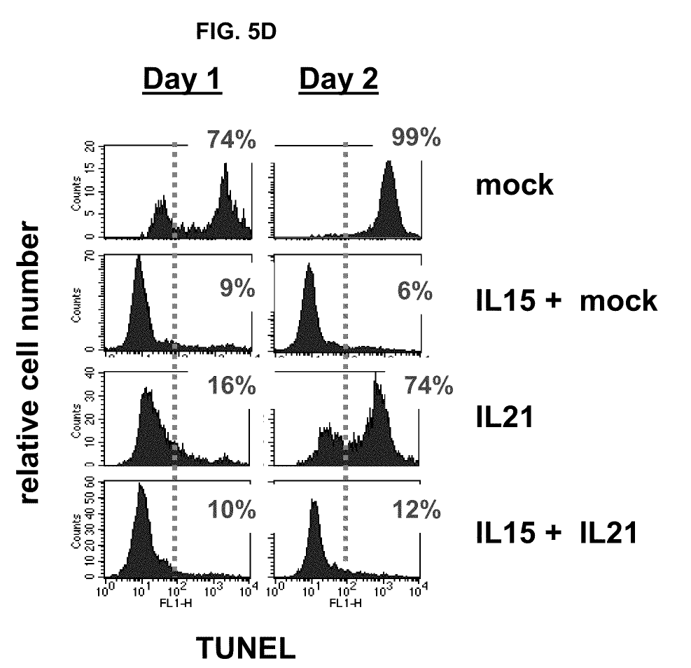

In addition to mediating cytotoxicity, activated NK cells produce IFNγ in an IL-12-dependent manner. In order to determine whether IL-21 treatment of activated NK cells affected IFNγ production, spleen cells stimulated in vitro for 7 days with IL-15 were re-challenged for 2 days with IL-15 and/or IL-21. Treatment of activated NK cells with IL-21 greatly enhanced IL-12-driven IFNγ production, and the response was further potentiated by the combination of IL-15 and IL-21 (FIG. 5A). In addition to boosting IL-12-dependent IFNγ production, IL-21 treatment also resulted in high levels of IFNγ production in the absence of added IL-12 (FIG. 5A). In contrast, when cells from IL-21R−/− mice were activated with IL-15 then challenged with IL-21, no enhanced spontaneous or IL-12-driven IFNγ production was found (FIG. 5B).

Thus, previously stimulated, but not resting, NK cells showed strong induction of cytotolytic activity (FIG. 4A-C) and IFNγ production (FIG. 5A) when exposed to IL-21. Experiments using FACS-sorted populations of >95% pure NK and T cells confirmed that both activities could be attributed almost exclusively to NK cells in these cultures. Interestingly, however, enhanced effector responses were not accompanied by growth effects. Cultures of IL-15-stimulated NK cells that were re-challenged for 2 days with IL-21 contained fewer NK cells than those maintained IL-15 (FIG. 5C). Examination of these cultures after 5 days of challenge confirmed that IL-21 not only failed to sustain NK viability but, when used in combination with IL-15, IL-21 reduced NK cell survival mediated by that cytokine (FIG. 5C). This was seen at all doses of IL-15 to which the cells responded (FIG. 3D). Thus, IL-21 boosted the effector functions of activated NK cells, but did not promote their viability, such that although activity per cell was increased, their number was sharply reduced.

Because of its effects on viability, the ability of IL-21 to directly induce NK cell death by apoptosis was examined. The TUNEL staining method was used on IL-15-expanded cultures restimulated for 2 days with IL-15, IL-21, or COS mock control supernatant. In cultures treated with COS mock control, most NK cells were apoptotic within one day, indicating that apoptosis occurs rapidly upon withdrawal of IL-15. As compared to COS mock control, IL-21 delayed the apoptosis caused by removal of IL-15. Nevertheless, after 2 days of restimulation with IL-21, the majority of NK cells in the culture were apoptotic (FIG. 5D). Taken together with observations that cells in similarly treated cultures restimulated for 2 days with IL-21 displayed high levels of cytotolytic activity (FIG. 5C) and IFNγ production (FIG. 5A), these findings indicate IL-21 induces high levels of effector function in NK cells undergoing apoptosis. Restimulation with IL-15+IL-21 also resulted in enhanced NK cell effector function (FIGS. 5C and 5A), but prevented or delayed apoptosis (FIG. 5D). This indicates that apoptosis is not a necessary correlate of the IL-21-mediated enhancement of NK cell effector function.

### Example 6

### IL-21 Blocks IL-15-Dependent Expansion of CD44^{hi} CD8+ TCR-Independent T Cells and T Cell Cytokine Receptor Expression

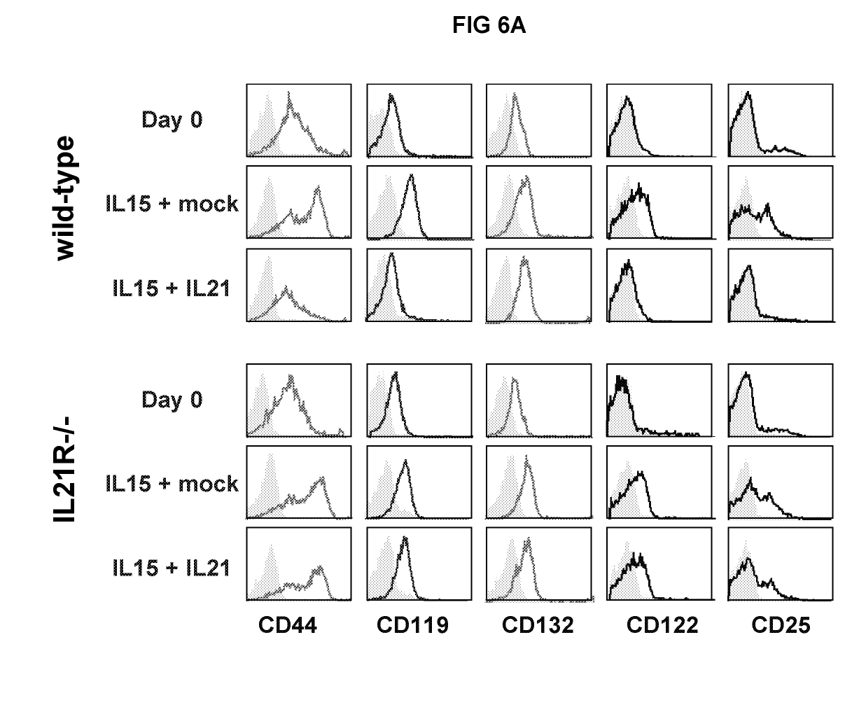

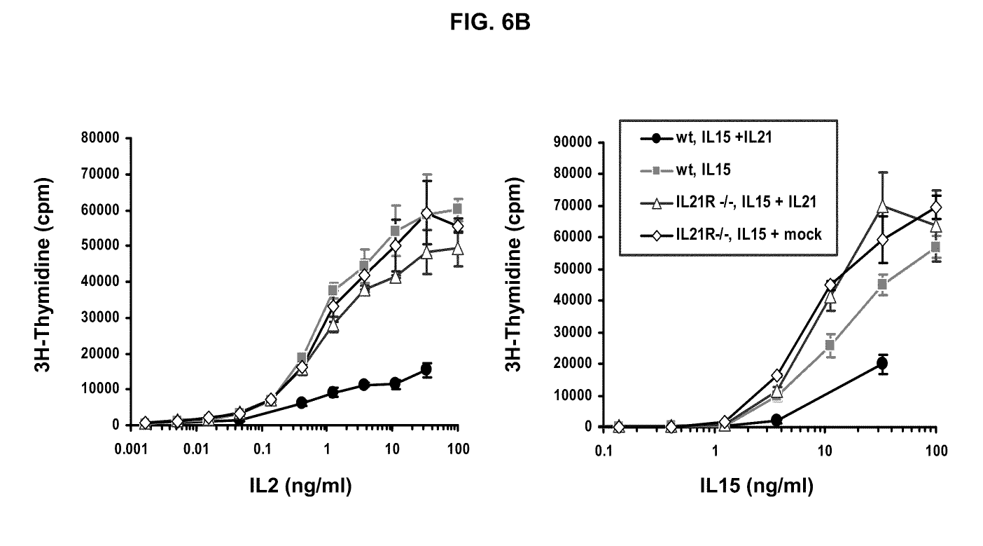

In the mouse, IL-15 in the absence of a TCR signal induces proliferation of CD8+ T cells expressing high levels of CD44, corresponding to a “memory” phenotype (Zhang et al., 1998; Sprent et al., Current Opin. Immunol. 13: 248-254, 2001). In accordance with this, IL-15-expanded spleen CD8+ T cells from either wild-type or IL-21R−/− mice were skewed toward high level expression of CD44 (FIG. 6A). Addition of IL-21 counteracted the expansion of CD44^{hi} CD8+ T cells from wild-type mice, but had no effect on cells from IL-21R−/− mice (FIG. 6A). Because TCR-independent CD44^{hi} CD8+ T cells are responsive to IFNγ in addition to IL-15 (Tough et al., J. Immunol. 166: 6007-6011: 2001), expression of the IFNγ receptor, CD119, was also examined. IL-21 also prevented the expansion of cells expressing this marker in response to IL-15 on cells from wild-type, but not IL-21R−/− mice (FIG. 6A).

To further examine IL-21 effects on the cytokine-responsiveness of T cells expanded with IL-15, the levels of CD25 (IL-2R α), CD122 (shared β chain of IL-2R and IL-15R), and CD132 (γc) was examined on spleen T cells following exposure to IL-15 in the presence or absence of IL-21. Both CD25 and CD122 expression was increased upon IL-15 treatment of cells from wild-type and IL-21R−/− mice. Addition of IL-21 prevented this receptor induction on T cells from wild-type mice, but had no effect on cells from IL-21R−/− mice (FIG. 6A). Expression of γc (CD132) was not affected by IL-21 (FIG. 6A). The decreased expression of receptor chains suggested that in the presence of IL-21, the responsiveness of splenic T cells to IL-2 or IL-15 would be reduced. In accordance with this, wild-type spleen cells that had been expanded with IL-15 in the presence of IL-21 showed less proliferation in response to IL-2 or IL-15 than those maintained in the absence of IL-21. Cells from IL-21R−/− mice were unaffected by IL-21 (FIG. 12B). Thus, IL-21 prevented IL-15-driven, antigen-independent T cell responses, including the expansion of CD44^{hi}CD8+ cells and the increased expression of functional cytokine receptors.

### Example 7

### IL-21 Enhances T Cell Responses to Allo-Antigen

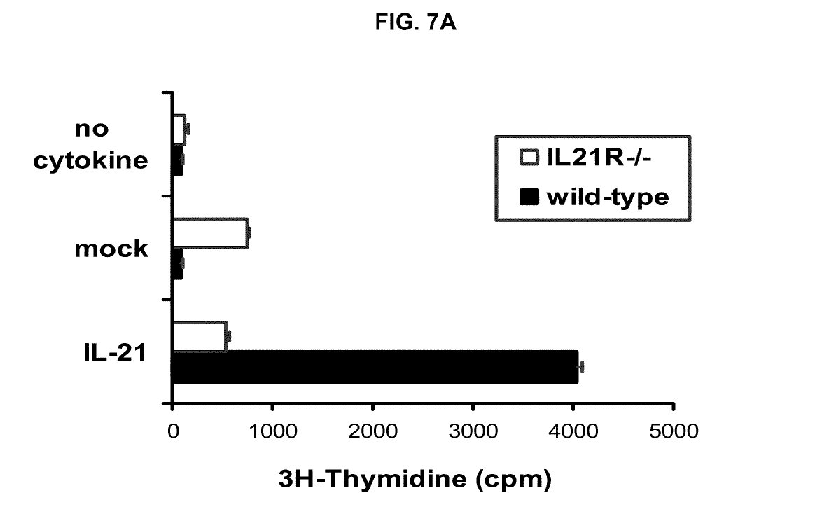

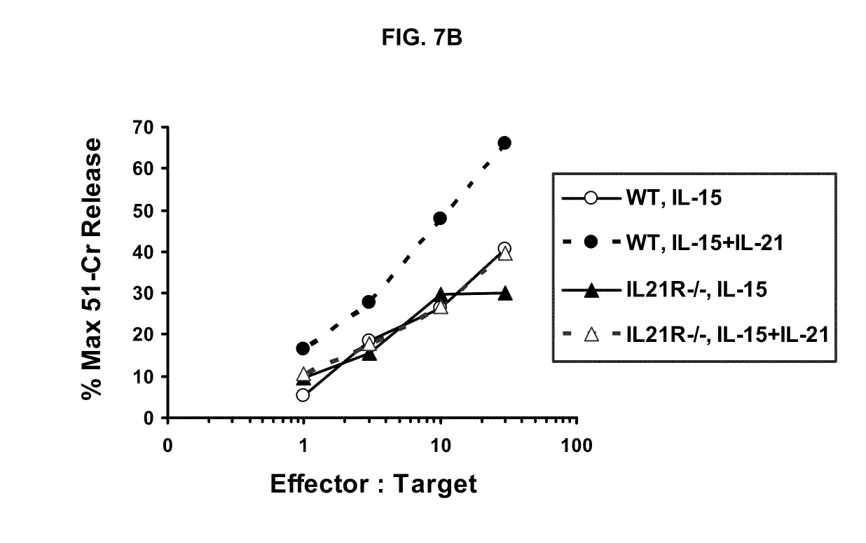

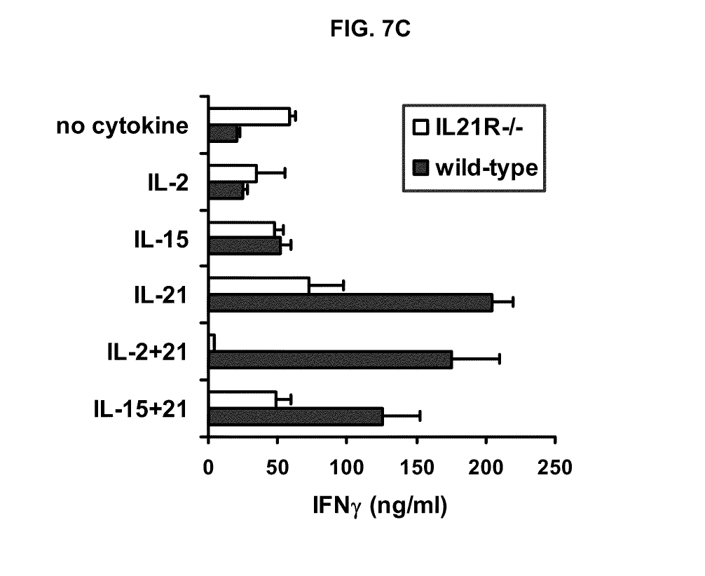

The effect of IL-21 in an antigen-driven T cell response was examined using a mixed lymphocyte reaction system. Purified lymph node T cells from wild-type or IL-21R−/− (H-2^{b/d}) mice were activated for 3-5 days with irradiated allogeneic splenocytes (H-2^{k}) in the presence of IL-21 or control supernatant. Similar to results with anti-CD3 stimulation (FIG. 7C), IL-21 enhanced alloantigen stimulation of wild-type, but not IL-21R−/− T cells (FIG. 7A). T cells from both IL-21R−/− and wild type mice exhibited enhanced proliferation to alloantigen in the presence of IL-2 or IL-15 and thus have no intrinsic defects in responsiveness. Stimulation of T cells results in the development of effector functions, including CTL activity and IFNγ production. Therefore, we compared the ability of IL-21R−/− and wild type T cells to differentiate into allo-specific effectors and examined the effects of IL-21 and related cytokines on this process. T cells from wild-type or IL-21R−/− mice primed with alloantigen and IL-15 displayed strong CTL activity towards allo-specific target cells (FIG. 7B). Priming in the presence of IL-15+IL-21 further enhanced the development of lytic activity in wild-type, but not IL-21R−/− cultures, indicating that IL-15 and IL-21 cooperatively enhance CTL differentiation. Similar results were observed when cells were primed in the presence of IL-2. IL-21 added in the absence of other exogenous cytokines also enhanced the development of allo-specific CTL activity from wild-type cells; however, the addition of IL-15 or IL-2 was necessary to generate sufficient numbers of IL-21R−/− cells to perform these assays. After priming with allogeneic APCs and the indicated cytokines, wild-type or IL-21R−/− T cells were restimulated and IFNγ production was determined as another measure of effector function. Wild-type T cells primed in the presence of IL-21, alone or in combination with IL-2 or IL-15, secreted higher titers of IFNγ compared to those primed with IL-2 or IL-15 alone (FIG. 7C). Taken together, these results suggest that IL-21 enhances in vitro T cell responses to alloantigen in primary stimulation, and results in the generation of more potent effector T cells.

Additional embodiments are within the claims.

What is claimed is:

1. A transgenic non-human mammal whose genome comprises a disruption of an IL-21 receptor (IL-21R) gene such that the mammal lacks or has reduced levels of functional IL-21 receptor polypeptide.

2. The transgenic mammal of claim 2, wherein thymocytes from said transgenic mammal do not proliferate when contacted with IL-21.

3. The transgenic mammal of claim 2, wherein the mammal is a rodent.

4. The rodent of claim 3, wherein said rodent is a mouse.

5. The transgenic mammal of claim 4, wherein said IL-21R gene encodes a IL-21R polypeptide comprising the amino acid sequence of SEQ ID NO:2.

6. The transgenic mammal of claim 1, wherein one allele of the IL-21R gene in said mammal is disrupted.

7. The transgenic mammal of claim 1, wherein two alleles of the IL-21R gene in said mammal is disrupted.

8. The transgenic mammal of claim 1, wherein the disruption of the IL-21R gene is located on a homologue of human chromosome 16p12.

9. The transgenic mammal of claim 1, wherein the disruption of the IL-21 R gene comprises a substitution of an exon of said IL-21R gene with an exogenous nucleic acid sequence.

10. A cultured cell isolated from the transgenic mammal of claim 1, wherein the genomes of the cells comprise a disruption of a IL-21R gene.

11. An isolated mammalian cell whose genome comprises a disruption of an IL-21 receptor (IL-21R) gene such that the cell lacks or has reduced levels of functional IL-21 receptor polypeptide.

12. The isolated cell of claim 11, wherein said cell is an embryonic stem cell.

13. The embryonic stem cell of claim 12, wherein said embryonic stem cell is a murine embryonic stem cell.

14. The murine embryonic stem cell of claim 13, wherein murine stem cell is derived from a mouse strain of C57BL/6 origin.

15. The murine embryonic stem cell of claim 14, wherein said stem cell is a J12 embryonic stem cell.

16. A method of producing a non-human mammal with a disruption in a IL-21 receptor (IL-21R) gene, the method comprising:
introducing a transgenic non-human embryonic stem cell whose genome comprises a disruption of an IL-21 receptor (IL-21R) gene such into a blastocyst, thereby forming a chimeric blastocyst;
introducing the chimeric blastocyst into the uterus of a pseudopregnant mammal; and
recovering at least one transgenic progeny from said pseudopregnant mammal, wherein the genome of said progeny comprises a disruption of the IL-21 R gene such that the progeny lacks or has reduced levels of functional IL-21 R polypeptide.

17. The method of claim 16, wherein said transgenic non-human embryonic stem cell is prepared by
introducing a targeting vector which disrupts the IL-21R gene into a mammalian embryonic stem cell, thereby producing a transgenic embryonic stem cell with the disrupted IL-21R gene; and
selecting the transgenic embryonic stem cell whose genome comprises the disrupted IL-21R gene.

18. The method of claim 17, further comprising:
breeding the transgenic mammal with a second mammal to generate F1 progeny having a heterozygous disruption of the IL-21R gene, thereby expanding the population of mammals having a heterozygous disruption of the IL-21Rgene; and
crossbreeding the F1 progeny to produce a transgenic mammal that contains a homozygous disruption of the IL-21R gene.

19. The transgenic mammal of claim 18, wherein the mammal is a rodent.

20. A method for identifying the role of IL-21 in a biological process, the method comprising
providing a transgenic cell whose genome comprises a disruption of an IL-21 receptor (IL-21R) gene such that the cell lacks or has reduced levels of functional IL-21 receptor polypeptide;
measuring one or more properties associated with said biological process; and
comparing said one or more properties to a reference cell whose genome does not have a disruption in an IL-21R gene,
wherein a difference in said one or more properties indicates IL-21 affects said biological process.

21. A method for identifying the role of IL-21 in a biological process, the method comprising
providing a transgenic non-human mammal whose genome comprises a disruption of an IL-21 receptor (IL-21R) gene such that the cell lacks or has reduced levels of functional IL-21 receptor polypeptide;
measuring one or more properties associated with said biological process; and
comparing said one or more properties to a reference mammal whose genome does not have a disruption in an IL-21R gene,
wherein a difference in said one or more properties indicates IL-21 affects said biological process.

22. A method for determining whether a test agent selectively modulates IL-21 receptor (IL-21R) activity, the method comprising:
administering a test agent to a first non-human mammal and a second non-human mammal, wherein said first non-human mammal comprises functional wild-type IL-21R polypeptide and wherein said the genome of said second non-human transgenic mammal comprises a disruption of its endogenous IL-21R genes such that the mammal lacks functional IL-21R polypeptide;
comparing a biological response elicited said agent in said first mammal and said second mammal;
wherein an alteration in said response indicated test agent selectively modulates the IL-21 receptor.


SEQUENCE LISTING (3 sequences)

SEQ ID NO: 1 - DNA, 2598 bp, Mus musculus
cagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccagtggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataacaaaaaaaaaaaaaaa

SEQ ID NO: 2 - protein, 529 aa, Mus musculus
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 3 - protein, 16 aa, Mus musculus
MPRGPVAALLLLILHG